(19) United States
(12) Patent Application Publication
(10) Pub. No.: US 2010/0183529 A1
(43) Pub. Date: Jul. 22, 2010

(54) Cosmetic compositions comprising photostabilized dibenzoylmethane compounds and 2-pyrrolidinone-4- carboxy esters

(75) Inventors: Hervé Richard, Gagny (FR); Xavier Marat, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/654,031

(22) Filed: Dec. 8, 2009

Related U.S. Application Data
(60) Provisional application No. 61/193,646, filed on Dec. 12, 2008.

(30) Foreign Application Priority Data
Dec. 8, 2008 (FR) 0858335

(51) Int. Cl. A61K 8/49; A61Q 17/04; A61K 8/58

(52) U.S. Cl. 424/60; 424/59

(57) ABSTRACT

Photostable cosmetic compositions contain, formulated into a cosmetically acceptable vehicle, at least one UV-screening system that includes:
(a) at least one dibenzoylmethane compound, and
(b) at least one 2-pyrrolidinone-4-carboxy ester compound having the following formula (I):

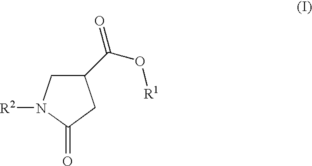

in which:
R¹ is a linear or branched C₁-C₂₀ alkyl radical, and
R² is a linear or branched C₁-C₂₀ alkyl radical which optionally includes a C₅-C₆ ring member, the phenyl radical, the benzyl radical or the phenethyl radical.

### CROSS-REFERENCE TO PRIORITY/PROVISIONAL APPLICATIONS

This application claims priority under 35 U.S.C. §119 of FR 0858335, filed Dec. 8, 2008 and §120 of U.S. Provisional Application No. 61/193,646, filed Dec. 12, 2008, each hereby expressly incorporated by reference and each assigned to the assignee hereof.

### BACKGROUND OF THE INVENTION

1. Technical Field of the Invention

The present invention relates to cosmetic compositions comprising the combination of at least one screening agent of the dibenzoylmethane compound type and at least one 2-pyrrolidinone-4-carboxy ester compound of formula (I), the definition of which is given below.

This invention also relates to a process for the photostabilization, with regard to radiation, of at least one screening agent of the dibenzoylmethane compound type by a specific 2-pyrrolidinone-4-carboxy ester compound of formula (I), the definition of which is also given below.

The present invention also relates to the formulation of at least one specific 2-pyrrolidinone-4-carboxy ester compound of formula (I) into a composition comprising, in a cosmetically acceptable vehicle, at least one dibenzoylmethane compound for the purpose of improving the effectiveness of the said composition with regard to UV-A rays.

2. Description of Background and/or Related and/or Prior Art

It is known that light radiation with wavelengths of from 280 nm to 400 nm makes possible browning of the human epidermis and that rays with wavelengths more particularly of from 280 to 320 nm, known as UV-B, cause erythemas and skin burns which may be harmful to the development of natural tanning. For these reasons, and for aesthetic reasons, there exists a constant demand for means for controlling this natural tanning for the purpose of thus controlling the color of the skin; it is thus advisable to screen out this UV-B radiation.

It is also known that UV-A rays, with wavelengths of from 320 and 400 nm, which cause browning of the skin, are capable of bringing about a detrimental change in the latter, in particular in the case of sensitive skin or of skin continually exposed to solar radiation. UV-A rays cause in particular a loss in the elasticity of the skin and the appearance of wrinkles, resulting in premature skin aging. They promote the triggering of the erythemal reaction or accentuate this reaction in certain subjects and can even be the cause of phototoxic or photoallergic reactions. Thus, for aesthetic and cosmetic reasons, such as the preservation of the natural elasticity of the skin, for example, increasingly people desire to control the effect of UV-A rays on their skin. It is therefore desirable also to screen out UV-A radiation.

With the goal of providing protection of the skin and keratinous substances against UV radiation, use is generally made of anti-sun compositions comprising organic screening agents which are active in the UV-A region and which are active in the UV-B region. The majority of these screening agents are fat-soluble.

In this respect, a particularly advantageous family of UV-A screening agents is currently composed of dibenzoylmethane compounds and in particular 4-tert-butyl-4's-methoxydibenzoylmethane, this because these exhibit a high intrinsic absorption power. These dibenzoylmethane compounds, which are now products well known per se as screening agents active in the UV-A region, are described in particular in FR-A,326,405 and FR-A,2,440,933, and in EP-A,0,114,607; furthermore, 4-tert-butyl-4's-methoxydibenzoylmethane is currently offered for sale under the trademark “Parsol 1789®” by DSM Nutritional Products.

Unfortunately, it is found that dibenzoylmethane derivatives are products which are relatively sensitive to ultraviolet radiation (in particular UV-A radiation), that is to say, more specifically, that they exhibit an unfortunate tendency to decompose more or less rapidly under the action of the latter. Thus, this substantial lack of photochemical stability of dibenzoylmethane compounds in the face of the ultraviolet radiation to which they are by nature intended to be subjected does not make it possible to guarantee continuous protection during prolonged exposure to the sun, such that repeated applications at regular and close intervals of time have to be carried out by the user to obtain effective protection of the skin against UV rays.

It is known that, in EP,717,982, amide compounds have a photostabilizing effect on dibenzoylmethane compounds and more particularly N,N-disubstituted amidated oils, such as the compound N,N-diethyl-3-methylbenzamide having the structure:

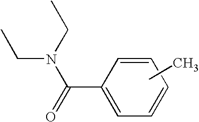

or ethyl N-butyl-N-acetylaminopropionate of formula:

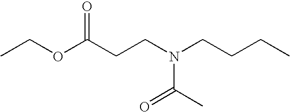

such as the product marketed under the trademark R3535 by Merck. These amidated oils have been described in particular in U.S. Published Patent Application 2007/141014 as solvents in cosmetic formulations of active principles which are difficult to dissolve in oils, such as UV-screening agents, flavone derivatives, chromone derivatives, aryloximes and parabens.

However, this stabilization is obtained in the presence of 20 to 30% of the latter compound, which exhibits a high solvating power for all the starting materials involved in producing the formulations, the consequence of which is reflected by a destabilization of the compositions, rendering unsuitable for use the compositions comprising such a combination.

U.S. Pat. No. 6,528,068 also discloses sun compositions comprising amidated oils which are neutral esters of N-acylamino acid comprising a linear or branched long-chain C₆-C₂₂ acyl group, such as Isopropyl Lauroyl Sarcosinate (Eldew SL 205 from Ajinomoto), in combination with organic UV-screening agents which are difficult to dissolve in the oils commonly included in sun formulations. The combination of these amidated oils with a dibenzoylmethane compound, such as 4-tert-butyl-4's-methoxydibenzoylmethane, does not make it possible to provide a completely satisfactory dibenzoylmethane photostability.

The photostabilization of dibenzoylmethane compounds with regard to UV radiation by amidated compounds thus constitutes, at the present time, a problem which has not yet been completely satisfactorily solved.

### SUMMARY OF THE INVENTION

It has now surprisingly been discovered that by combining the abovementioned dibenzoylmethane compounds with specific 2-pyrrolidinone-4-carboxy ester compounds of formula (I) which will be defined in detail below, it is possible to further improve, substantially and markedly, the photochemical stability (or photostability) of these same dibenzoylmethane compounds and their effectiveness in the UV-A region. The compositions comprising such a combination also result, after application, in a more homogeneous distribution of the dibenzoylmethane screening agent.

This discovery provides the basis of the present invention.

Thus, in accordance with the present invention, compositions are now provided comprising, formulated into a cosmetically acceptable vehicle, at least one UV-screening system which comprises:

(a) at least one dibenzoylmethane compound and

(b) at least one 2-pyrrolidinone-4-carboxy ester compound of formula (I), the definition of which is given below.

The present invention also features a method for improving the chemical stability with regard to UV radiation of at least one dibenzoylmethane compound which entails combining the said dibenzoylmethane compound with an effective amount of at least one 2-pyrrolidinone-4-carboxy ester compound of formula (I), the definitions of which is given below.

Another aspect of the present invention is the formulation of at least one 2-pyrrolidinone-4-carboxy ester compound of formula (I) into a composition comprising, in a cosmetically acceptable vehicle, at least one dibenzoylmethane compound with the goal of improving the effectiveness of the said composition with regard to UV-A radiation.

Other characteristics, aspects and advantages of the invention will become apparent from the detailed description which follows.

“Cosmetically acceptable” means compatible with the skin and/or its superficial body growths, exhibiting a pleasant color, a pleasant odor and a pleasant feel, and not causing unacceptable discomfort (smarting, tightness, redness) liable to dissuade the consumer from using this composition.

“Effective amount” means an amount sufficient to produce a notable and significant improvement in the photostability of the dibenzoylmethane compound(s) in the cosmetic composition. This minimum amount of 2-pyrrolidinone-4-carboxy ester compound of formula (I), which can vary according to the nature of the vehicle selected for the composition, can be determined without any difficulty by means of a conventional test for measuring photostability, such as that provided in the examples below.

### DETAILED DESCRIPTION OF BEST MODE AND SPECIFIC/PREFERRED EMBODIMENTS OF THE INVENTION

The 2-pyrrolidinone-4-carboxy ester compounds in accordance with the invention are selected from those corresponding to the following general formula (I):

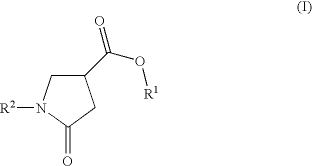

in which:

R¹ is a linear or branched C₁-C₂₀ alkyl radical,

R² is a linear or branched C₁-C₂₀ alkyl radical which can contain a C₅-C₆ ring, the phenyl radical, the benzyl radical or the phenethyl radical. In the formula (I), particularly exemplary radicals include methyl, ethyl, n-propyl, isopropyl, n-butyl, isobutyl, tert-butyl, n-octyl, 2-ethylhexyl, dodecyl, hexadecyl, cyclohexyl and methylcyclohexyl radicals.

Among the compounds of formula (I), particularly exemplary are the following compounds (a) to (oo):

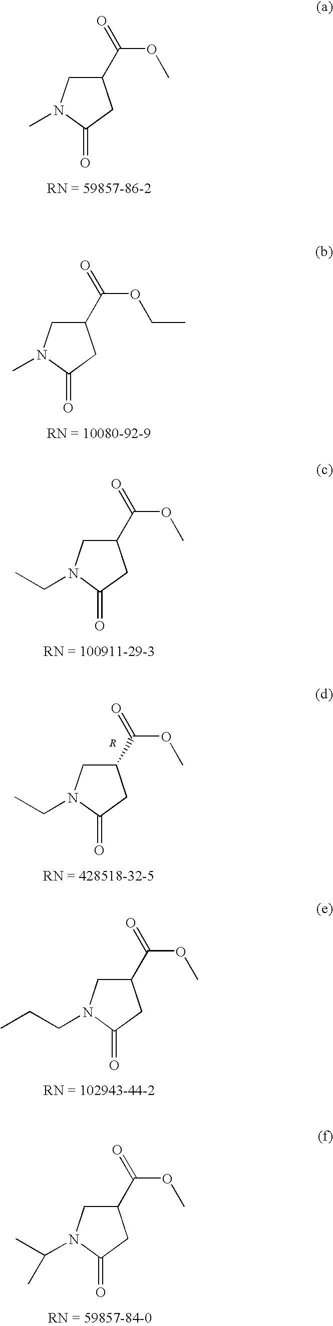

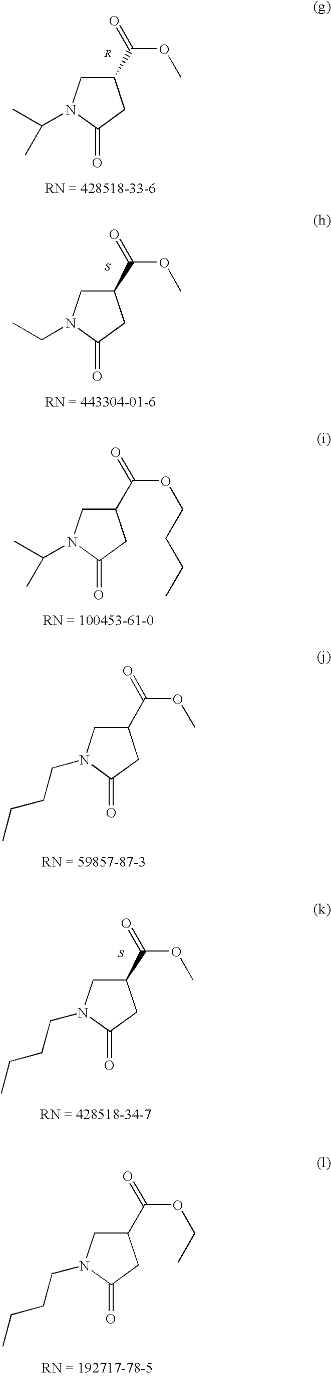

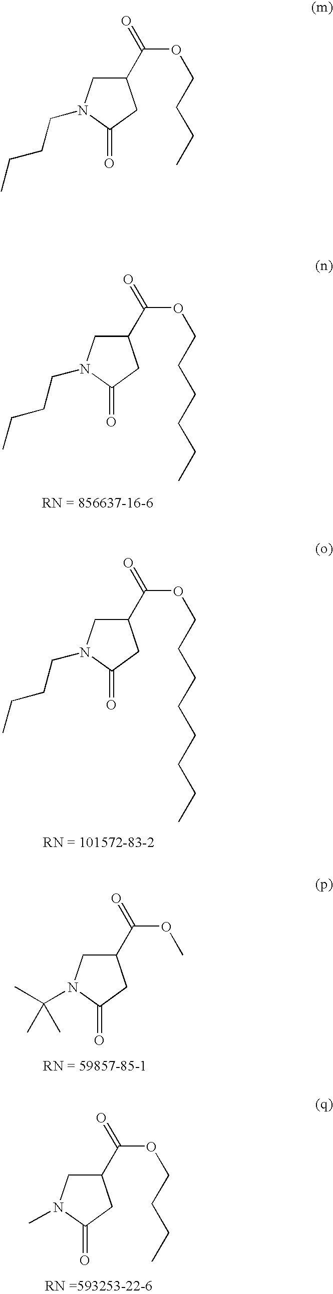

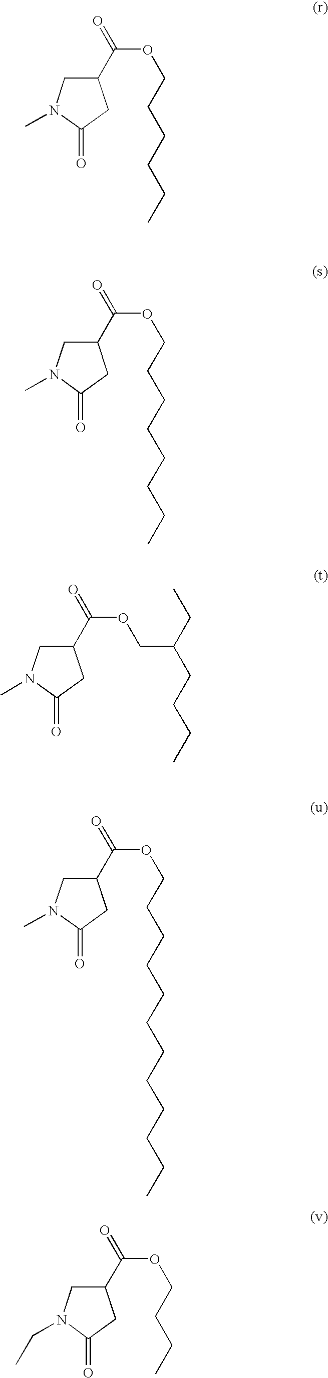

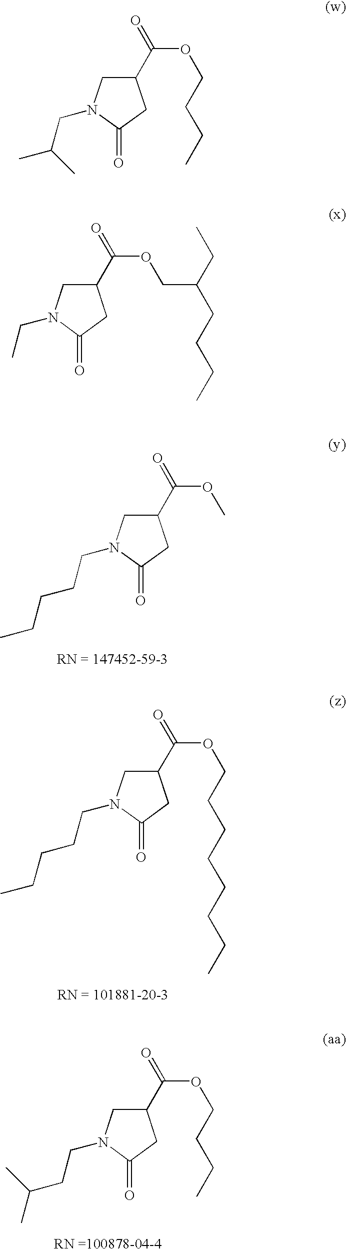

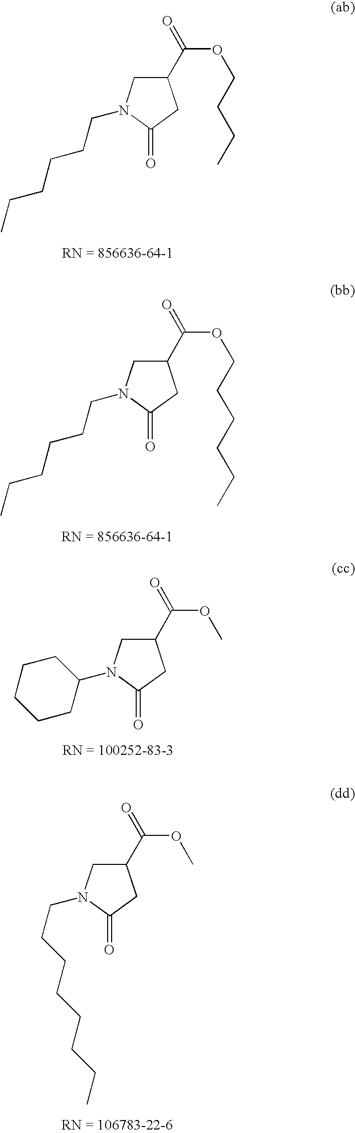

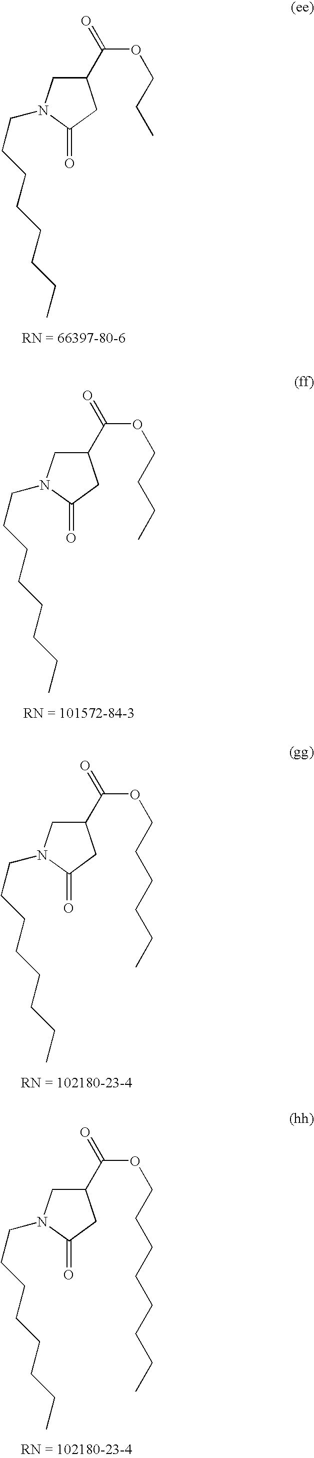

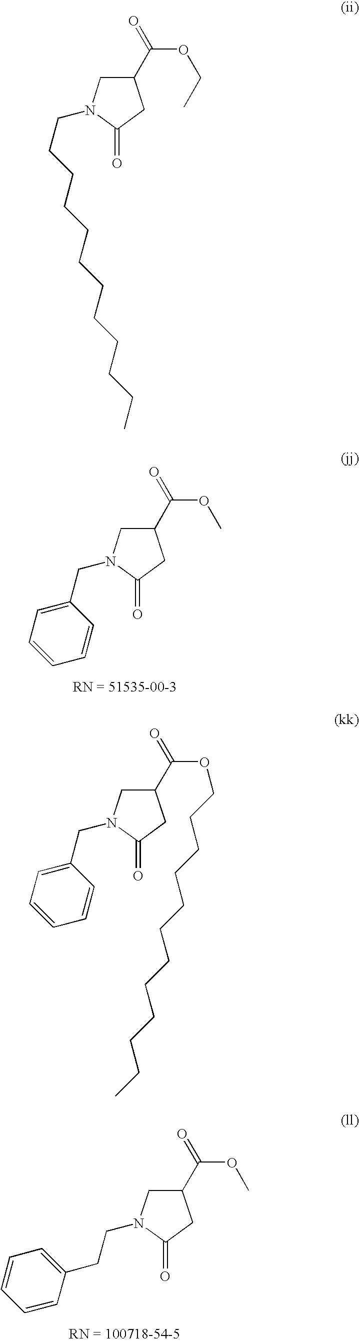

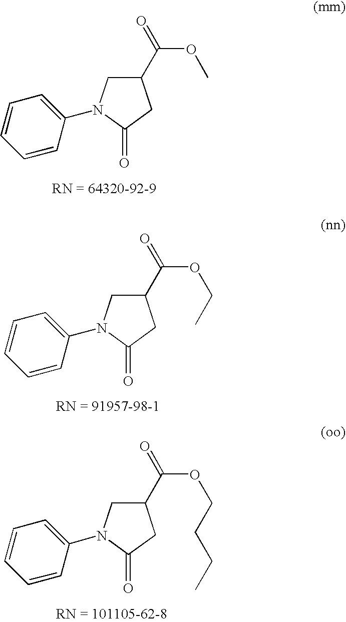

More particularly preferred are the compounds (j), (l), (m) and (n).

The compounds of formula (I), the syntheses of which are described in the following papers: J. Org. Chem., 26, pages 1519-24 (1961); Tetrahedron Asymmetric, 12 (23), pages 3241-9 (2001); J. Industrial & Engineering Chem., 47, pages 1572-8 (1955); J. Am. Chem. Soc., 60, pages 402-6 (1938); and in EP-0,069,512, U.S. Pat. No. 2,811,496 (1955), U.S. Pat. Nos. 2,826,588, 3,136,620, FR-2,290,199 and FR-2,696,744, can be easily prepared:

either by the condensation of a diester of itaconic acid of formula (II) with a primary amine of formula (III), with or without solvent, at a temperature of from 20° C. to 150° C., according to the following reaction scheme:

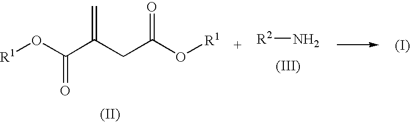

or in 2 stages starting from itaconic acid of formula (IV) by condensation with the primary amine of formula (III) in or not in the presence of a solvent, to give the intermediate acid of formula (V), followed by an esterification of this acid of formula (V) in the presence of an excess of alcohol of formula (VI), according to the following reaction scheme:

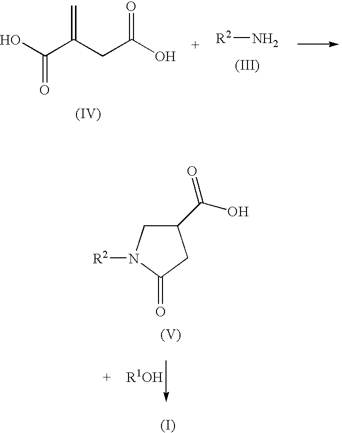

The 2-pyrrolidinone-4-carboxy ester compounds of formula (I) in accordance with the invention are preferably present in the compositions at contents of 0.01 to 20% by weight and more preferably of 0.1 to 10% by weight, with respect to the total weight of the composition.

Particularly exemplary dibenzoylmethane compounds are the following, without implying any limitation:
2-methyldibenzoylmethane,
4-methyldibenzoylmethane,
4-isopropyldibenzoylmethane,
4-(tert-butyl)dibenzoylmethane,
2,4-dimethyldibenzoylmethane,
2,5-dimethyldibenzoylmethane,
4,4's-diisopropyldibenzoylmethane,
4,4's-dimethoxydibenzoylmethane,
4-(tert-butyl)-4's-methoxydibenzoylmethane,
2-methyl-5-isopropyl-4's-methoxydibenzoylmethane,
2-methyl-5-(tert-butyl)-4's-methoxydibenzoylmethane,
2,4-dimethyl-4's-methoxydibenzoylmethane,
2,6-dimethyl-4-(tert-butyl)-4's-methoxydibenzoylmethane.

Among the abovementioned dibenzoylmethane compounds, one preferred species is 4-isopropyldibenzoylmethane, marketed under the trademark “Eusolex 8020” by Merck, which corresponds to the following formula:

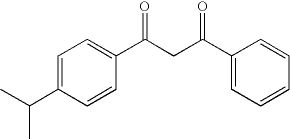

Also preferred is 1-(4-methoxy-1-benzofuran-5-yl)-3-phenylpropane-1,3-dione, marketed by Quest under the trademark Pongamol, of formula:

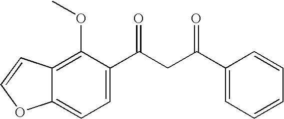

Very particularly preferred is 4-(tert-butyl)-4's-methoxydibenzoylmethane or Butyl Methoxy Dibenzoylmethane, marketed under the trademark “Parsol 1789” by DSM Nutritional Products; this screening agent has the following formula:

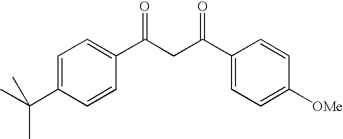

The dibenzoylmethane compound(s) can be present in the compositions in accordance with the invention at contents which preferably vary from 0.01 to 10% by weight and more preferably from 0.1 to 6% by weight, with respect to the total weight of the composition.

According to a particularly preferred embodiment of the invention, included in the compositions of the invention will additionally be a silicated s-triazine compound substituted by two aminobenzoate or aminobenzamide groups of following general formula (VI) or one of its tautomeric forms:

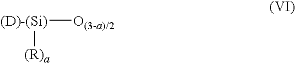

in which:

the radicals R, which may be identical or different, are each a linear or branched C₁-C₃₀ alkyl radical which is optionally halogenated or unsaturated, a C₆-C₁₂ aryl radical, a C₁-C₁₀ alkoxy radical, a hydroxyl radical or the trimethylsilyloxy group;

a=0 to 3; in addition to the structural units of formula -A-(Si)(R)ₐ(O)_{(3-a)/2}, the organosiloxane can comprise units of formula: (R)_{b}—(Si)(O)_{(4-b)/2}, in which:

R has the same definition as in the formula (II) and b=1, 2 or 3;

the group (D) is an s-triazine compound of following formula (VII):

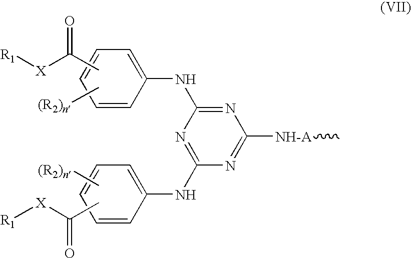

wherein:

X is —O— or —NR₃₋, with R₃ representing hydrogen or a C₁-C₅ alkyl radical,

R₁ is a linear or branched C₁-C₃₀ alkyl radical which is optionally unsaturated and which can comprise a silicon atom, a C₅-C₂₀ cycloalkyl radical, optionally substituted by 1 to 3 linear or branched C₁-C₄ alkyl radicals, the —(CH₂CHR₄—O)ₘR₆ group or the

CH₂—CH(OH)—CH₂—O—R₆ group,

R₄ is hydrogen or methyl; with the proviso that the (C═O)XR₁ group may be in the ortho, meta or para position with respect to the amino group,

R₅ is hydrogen or a C₁-C₈ alkyl radical,

R₆ is hydrogen or a C₄-C₈ alkyl radical,

m is an integer ranging from 2 to 20,

n's=0 to 2,

the radicals R₂, which may be identical or different, are each a hydroxyl radical, a linear or branched C₁-C₈ alkyl radical or a C₁-C₈ alkoxy radical, with the proviso that two adjacent R₂ groups on the same aromatic nucleus may together form an alkylidenedioxy group in which the alkylidene radical comprises 1 or 2 carbon atoms,

A is a divalent radical selected from methylene, —[CH(Si(CH₃)₃)]—, ethylene or a radical corresponding to one of the following formulae (VIII), (IX), (X) or (XI):

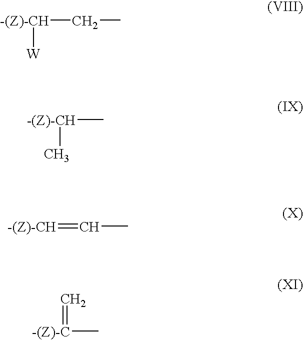

in which:

Z is a saturated or unsaturated and linear or branched C₁-C₁₀alkylene diradical which is optionally substituted by a hydroxyl radical or oxygen atoms and which can optionally comprise an amino group,

W is a hydrogen atom, a hydroxyl radical or a saturated or unsaturated and linear or branched C₁-C₈ alkyl radical.

It should be noted that the derivatives of formula (VI) can be in their tautomeric forms and more particularly in the tautomeric form of following formula (VI's):

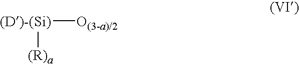

in which the group (D's) is an s-triazine compound of following formula (VII's):

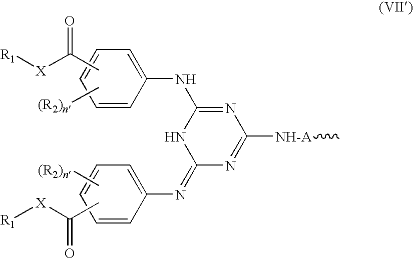

The preferred s-triazine derivatives are those for which, in the formula (VII) or (VIPs), at least one and more preferably all of the following characteristics are satisfied:

R is methyl,

a=1 or 2,

X is O,

R₁ is a C₄-C₅ radical,

n's=0,

the (C═O)XR₁ group is in the para position with respect to the amino group,

Z=—CH₂—,

W=H.

Preferably, the s-triazine compounds of the invention are represented by the following formulae (VIa), (VIb) and (VIc):

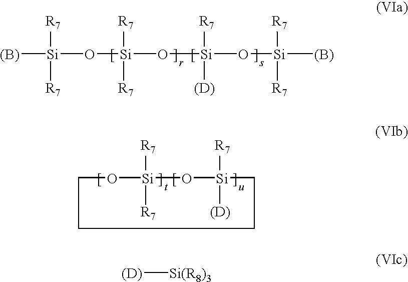

in which:

(D) corresponds to the formula (VII) as defined above,

the radicals R₇, which may be identical or different, are selected from linear or branched C₁-C₂₀ alkyl, phenyl, 3,3,3-trifluoropropyl and trimethylsilyloxy radicals or the hydroxyl radical,

the radicals R₈, which may be identical or different, are selected from linear or branched C₁-C₂₀ alkyl and alkenyl radicals, hydroxyl radicals or phenyl radicals,

the radicals (B), which may be identical or different, are selected from the R₇ radicals and the (D) radical,

r is an integer from 0 to 200, inclusive,

s is an integer ranging from 0 to 50 and, if s=0, at least one of the two (B) symbols is (D),

u is an integer ranging from 1 to 10,

t is an integer ranging from 0 to 10, with the proviso that t+u is equal to or greater than 3, and the tautomeric forms thereof.

The linear diorganosiloxanes of formula (VIa) are particularly preferred.

The linear or cyclic diorganosiloxanes of formula (VIa) or (VIb) within the scope of the present invention are random oligomers or polymers preferably exhibiting at least one or more preferably all of the following characteristics:

R₇ is the methyl radical or the hydroxyl radical,

B is preferably methyl (case of the linear compounds of formula (VIa)).

Particularly preferred compounds of formula (VI) are the compounds of following formulae (1) to (26) and their tautomeric forms:

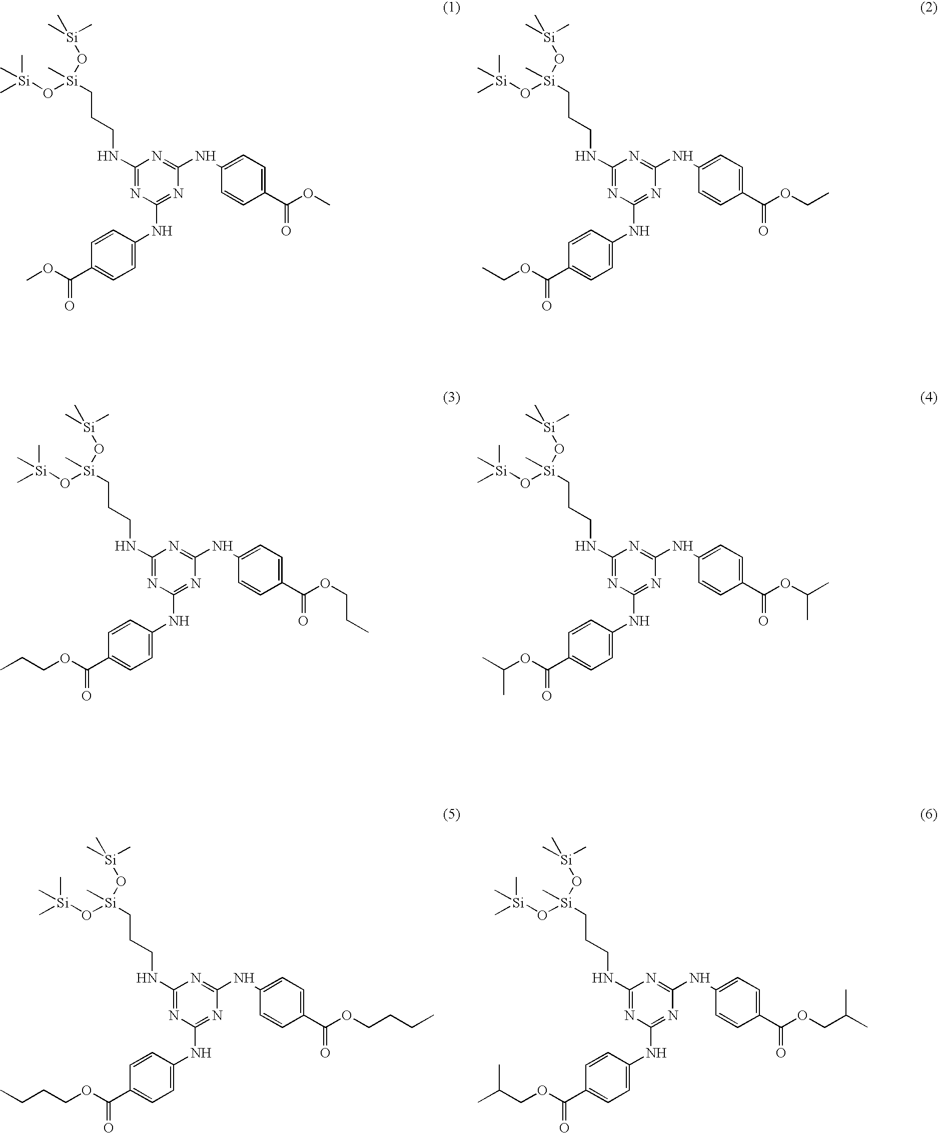

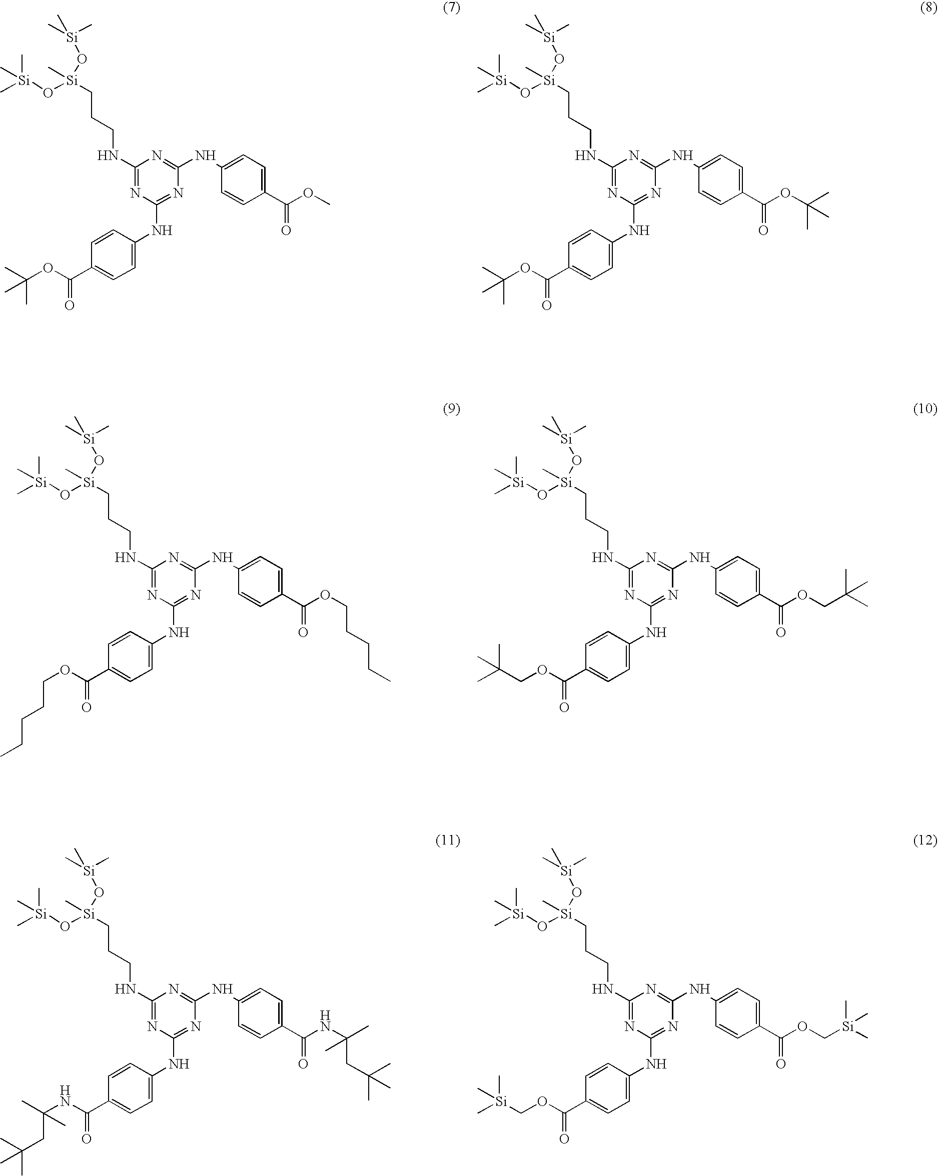

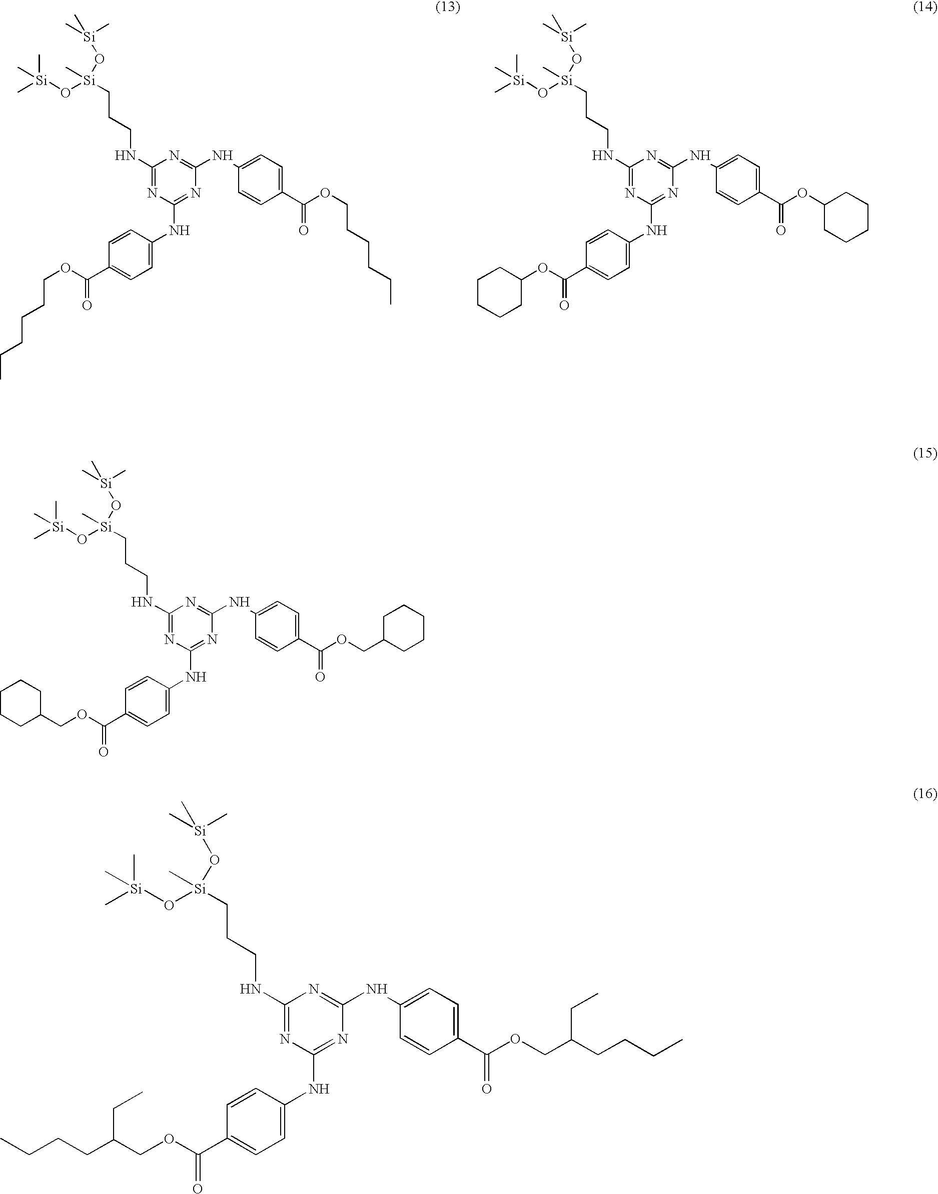

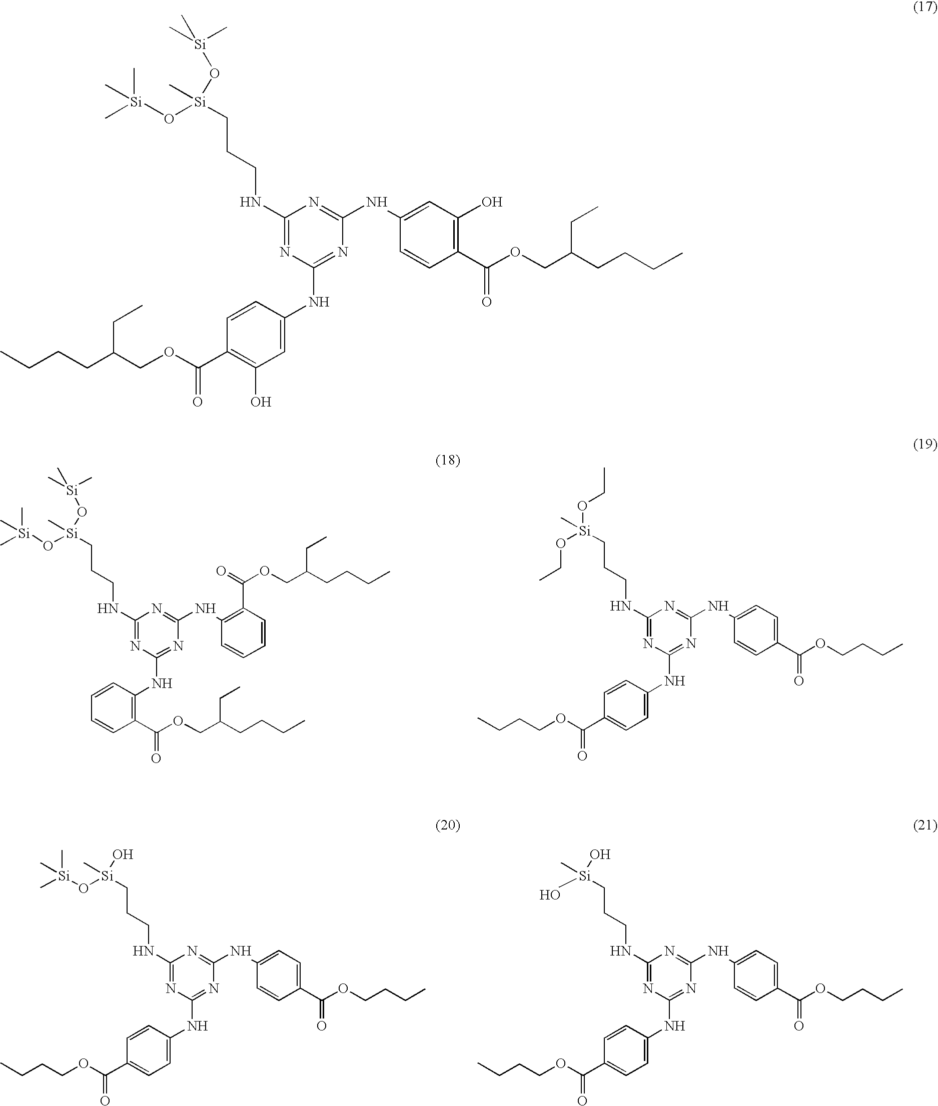

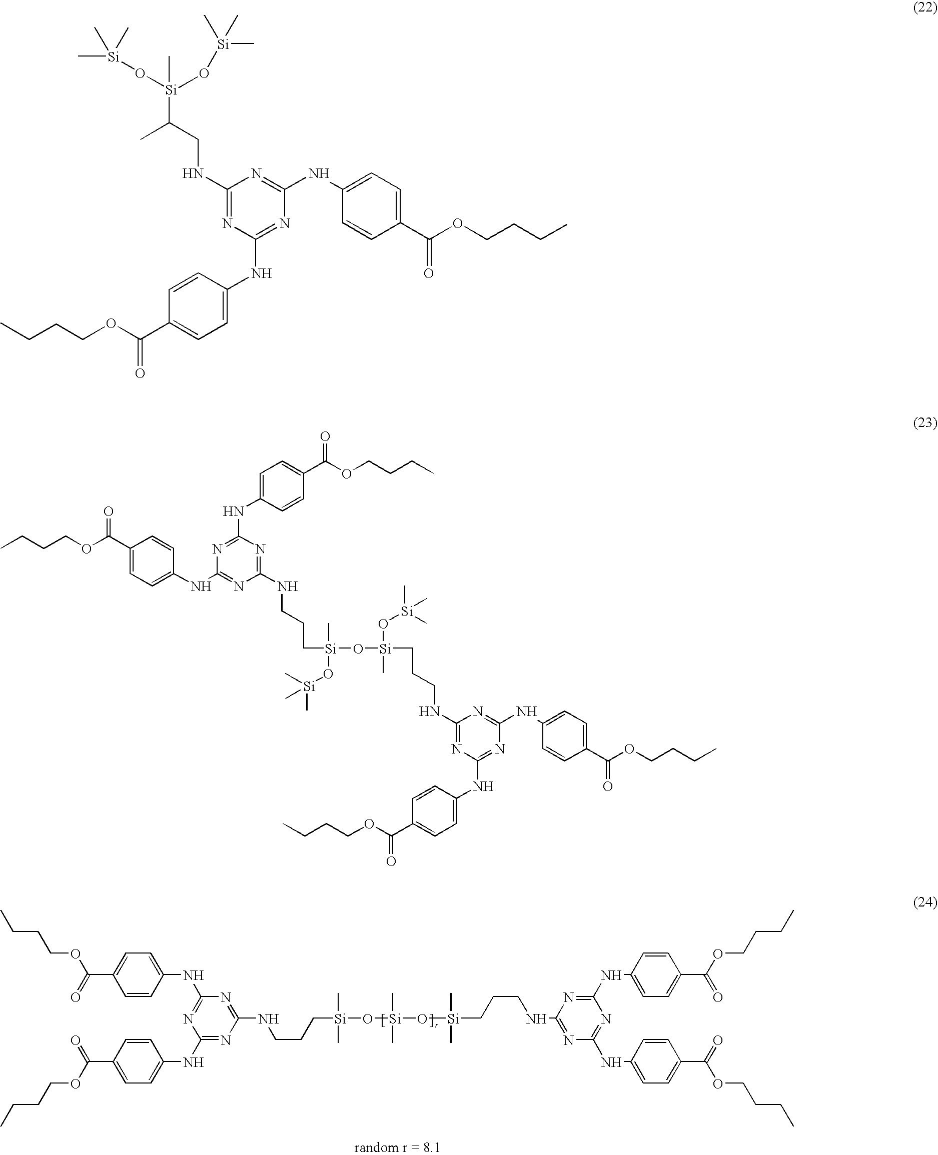

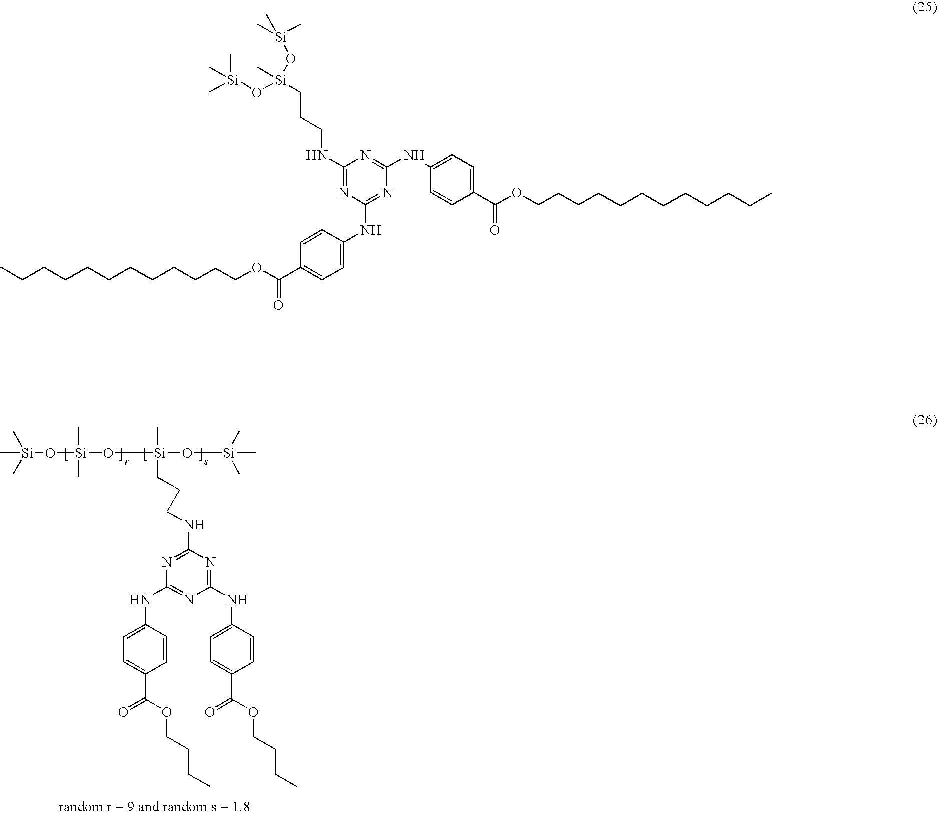

More particularly preferred are the compounds selected from:
2,4-bis(ethyl 4's-aminobenzoate)-6-[(3-{1,3,3,3-tetramethyl-1-[(trimethylsilyl)oxy]disiloxanyl}propyl)amino]-s-triazine of formula (2),

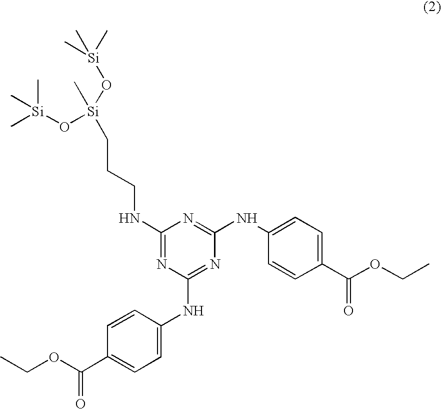

2,4-bis(isopropyl 4's-aminobenzoate)-6-[(3-{1,3,3,3-tetramethyl-1-[(trimethylsilyl)oxy]disiloxanyl}propyl)amino]-s-triazine of formula (4)

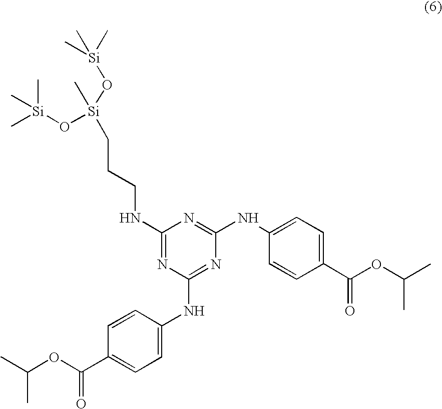

2,4-bis(n-butyl 4's-aminobenzoate)-6-[(3-{1,3,3,3-tetramethyl-1-[(trimethylsilyl)oxy]disiloxanyl}propyl)amino]-s-triazine of formula (5)

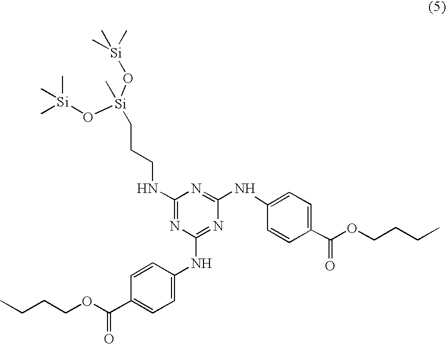

2,4-bis(isobutyl 4's-aminobenzoate)-6-[(3-{1,3,3,3-tetramethyl-1-[(trimethylsilyl)oxy]disiloxanyl}propyl)amino]-s-triazine of formula (6)

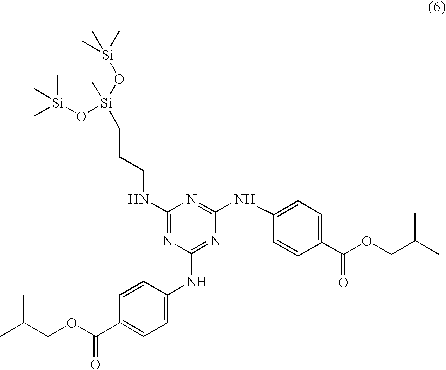

2,4-bis(neopentyl 4's-aminobenzoate)-6-[(3-{1,3,3,3-tetramethyl-1-[(trimethylsilyl)oxy]disiloxanyl}propyl)amino]-s-triazine of formula (10)

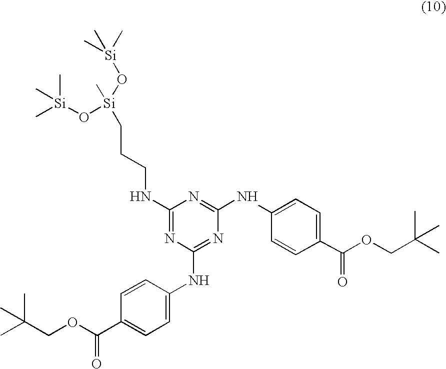

Still more particularly preferred is 2,4-bis(n-butyl 4's-aminobenzoate)-6-[(3-{1,3,3,3-tetramethyl-1-[(trimethylsilyl)oxy]disiloxanyl}propyl)amino]-s-triazine of formula (5):

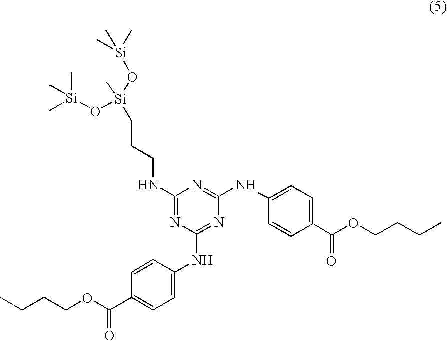

Certain triazine compounds of formula (II) and their tautomeric forms are known and have been described in EP-0,841,341 and FR-2,886,143.

The compositions according to the invention can additionally comprise other additional organic UV-screening agents active in the UV-A and/or UV-B regions which are water-soluble or fat-soluble or else insoluble in the cosmetic solvents commonly employed.

Of course, one skilled in the art will take care to select the optional additional screening agent or agents and/or their amounts such that the advantageous properties intrinsically associated with the compositions in accordance with the invention are not, or not substantially, detrimentally affected by the envisaged addition or additions, in particular the improvement in the photostability of the dibenzoylmethane derivative.

The additional organic screening agents are selected in particular from among anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives, in particular those cited in U.S. Pat. No. 5,624,663; benzimidazole derivatives; imidazolines; bis-benzoazolyl derivatives, such as described in EP-669,323 and U.S. Pat. No. 2,463,264; p-aminobenzoic acid (PABA) derivatives; methylenebis(hydroxyphenylbenzotriazole) derivatives, such as described in U.S. Pat. Nos. 5,237,071, 5,166,355, GB-2,303,549, DE-197,26,184 and EP-893,119; benzoxazole derivatives, such as described in EP-0,832,642, EP-1,027,883, EP-1,300,137 and DE-10162844; screening polymers and screening silicones, such as those described in particular in WO 93/04665; dimers derived from α-alkylstyrene, such as those described in DE-19855649; 4,4-diarylbutadienes, such as described in EP-0,967,200, DE-19746654, DE-19755649, EP-A-1,008,586, EP-1,133,980 and EP-133,981; merocyanine derivatives, such as those described in WO04006878, WO05058269 and WO06032741; and mixtures thereof.

Exemplary additional organic photoprotective agents are those noted below under their INCI names:

Cinnamic Derivatives:

Ethylhexyl Methoxycinnamate, marketed in particular under the trademark “Parsol MCX” by DSM Nutritional Products Inc.,

### Isopropyl Methoxycinnamate,

Isoamyl Methoxycinnamate, marketed under the trademark “Neo Heliopan E 1000” by Symrise,

### DEA Methoxycinnamate,

### Diisopropyl Methylcinnamate,

### Glyceryl Ethylhexanoate Dimethoxycinnamate,

para-Aminobenzoic Acid Derivatives:

### PABA,

### Ethyl PABA,

### Ethyl Dihydroxypropyl PABA,

Ethylhexyl Dimethyl PABA, marketed in particular under the trademark “Escalol 507” by ISP,

### Glyceryl PABA,

PEG-25 PABA, marketed under the trademark “Uvinul P25” by BASF,

Salicylic Derivatives:

Homosalate, marketed under the trademark “Eusolex HMS” by Rona/EM Industries,
Ethylhexyl Salicylate, marketed under the trademark “Neo Heliopan OS” by Symrise,
Dipropyleneglycol Salicylate, marketed under the trademark “Dipsal” by Scher,
TEA Salicylate, marketed under the trademark “Neo Heliopan TS” by Symrise,

β,β-Diphenylacrylate Derivatives:

Octocrylene, marketed in particular under the trademark “Uvinul N539” by BASF,
Etocrylene, marketed in particular under the trademark “Uvinul N35” by BASF,

Benzophenone Derivatives:

Benzophenone-1, marketed under the trademark “Uvinul 400” by BASF,
Benzophenone-2, marketed under the trademark “Uvinul D50” by BASF,
Benzophenone-3 or Oxybenzone, marketed under the trademark “Uvinul M40” by BASF,
Benzophenone-4, marketed under the trademark “Uvinul MS40” by BASF, Benzophenone-5,
Benzophenone-6, marketed under the trademark “Helisorb 11” by Norquay,
Benzophenone-8, marketed under the trademark “Spectra-Sorb UV-24” by American Cyanamid,
Benzophenone-9, marketed under the trademark “Uvinul DS-49” by BASF, Benzophenone-12,
n-Hexyl 2-(4-diethylamino-2-hydroxybenzoyl)benzoate, marketed under the trademark “Uvinul A+” by BASF,

Benzylidenecamphor Derivatives:

3-Benzylidene camphor, manufactured under the trademark “Mexoryl SD” by Chimex,
4-Methylbenzylidene camphor, marketed under the trademark “Eusolex 6300” by Merck,
Benzylidene Camphor Sulfonic Acid, manufactured under the trademark “Mexoryl SL” by Chimex,
Camphor Benzalkonium Methosulfate, manufactured under the trademark “Mexoryl SO” by Chimex,
Terephthalylidene Dicamphor Sulfonic Acid, manufactured under the trademark “Mexoryl SX” by Chimex,
Polyacrylamidomethyl Benzylidene Camphor, manufactured under the trademark “Mexoryl SW” by Chimex,

Phenylbenzimidazole Derivatives:

Phenylbenzimidazole Sulfonic Acid, marketed in particular under the trademark “Eusolex 232” by Merck,
Disodium Phenyl Dibenzimidazole Tetrasulfonate, marketed under the trademark “Neo Heliopan AP” by Symrise,

Phenylbenzotriazole Derivatives:

Drometrizole Trisiloxane, marketed under the trademark “Silatrizole” by Rhodia Chimie,
Methylene Bis-Benzotriazolyl Tetramethylbutylphenol, marketed in the solid form under the trademark “Mixxim BB/100” by Fairmount Chemical or in the micronized form in aqueous dispersion under the trademark “Tinosorb M” by Ciba Specialty Chemicals,

Triazine Derivatives:

Bis-Ethylhexyloxyphenol Methoxyphenyl Triazine, marketed under the trademark “Tinosorb S” by Ciba-Geigy,
Ethylhexyl Triazone, marketed in particular under the trademark “Uvinul T150” by BASF,
Diethylhexyl Butamido Triazone, marketed under the trademark “Uvasorb HEB” by Sigma 3V,
2,4,6-Tris(dineopentyl 4's-aminobenzalmalonate)-s-triazine,
2,4,6-Tris(diisobutyl 4's-aminobenzalmalonate)-s-triazine,
2,4-Bis(dineopentyl 4's-aminobenzalmalonate)-6-(n-butyl 4's-aminobenzoate)-s-triazine,
The symmetrical triazine screening agents described in U.S. Pat. No. 6,225,467, WO 2004/085412 (see compounds 6 and 9) or the document “Symmetrical Triazine Derivatives”, IP.COM Journal, IP.COM INC, WEST HENRIETTA, N.Y., US (20 Sep. 2004), in particular the 2,4,6-tris(biphenyl)-1,3,5-triazines (especially 2,4,6-tris(biphenyl-4-yl)-1,3,5-triazine) and 2,4,6-tris(terphenyl)-1,3,5-triazine, and also in WO 06/035000, WO 06/034982, WO 06/034991, WO 06/035007, WO 2006/034992 and WO 2006/034985.

Anthranilic Derivatives:

Menthyl anthranilate, marketed under the trademark “Neo Heliopan MA” by Symrise,

Imidazoline Derivatives:

### Ethylhexyl Dimethoxybenzylidene Dioxoimidazoline Propionate,

Benzalmalonate Derivatives:

Polyorganosiloxane comprising benzalmalonate functional groups, such as Polysilicone-15, marketed under the trademark “Parsol SLX” by DSM Nutritional Products Inc.,

4,4-Diarylbutadiene Derivatives:

1,1-Dicarboxy(2,2's-dimethylpropyl)-4,4-diphenylbutadiene,

Benzoxazole Derivatives:
2,4-bis[5-1(dimethylpropyl)benzoxazol-2-yl-(4-phenyl)imino]-6-(2-ethylhexyl)imino-1,3,5-triazine, marketed under the trademark Uvasorb K2A by Sigma 3V,

Merocyanine Derivatives:

Octyl 5-N,N-diethylamino-2-phenylsulphonyl-2,4-pentadienoate, and their mixtures.

The preferred organic screening agents are selected from:

Ethylhexyl Methoxycinnamate,

Ethylhexyl Salicylate,

Homosalate,

Octocrylene,

Phenylbenzimidazole Sulfonic Acid,

Benzophenone-3,

Benzophenone-4,

Benzophenone-5,

n-Hexyl 2-(4-diethylamino-2-hydroxybenzoyl)benzoate,

4-Methylbenzylidene Camphor,

Terephthalylidene Dicamphor Sulfonic Acid,

Disodium Phenyl Dibenzimidazole Tetrasulfonate,

Methylene Bis-Benzotriazolyl Tetramethylbutylphenol,

Bis-Ethylhexyloxyphenol Methoxyphenyl Triazine,

Ethylhexyl Triazone,

Diethylhexyl Butamido Triazone,

2,4,6-Tris(dineopentyl 4's-aminobenzalmalonate)-s-triazine,

2,4,6-Tris(diisobutyl 4's-aminobenzalmalonate)-s-triazine,

2,4-Bis(n-butyl 4's-aminobenzoate)-6-(aminopropyltrisiloxane)-s-triazine,

2,4-Bis(dineopentyl 4's-aminobenzalmalonate)-6-(n-butyl 4's-laminobenzoate)-s-triazine,

2,4,6-Tris(biphenyl-4-yl)-1,3,5-triazine,

2,4,6-Tris(terphenyl)-1,3,5-triazine,

Drometrizole Trisiloxane,

Polysilicone-15,

1,1-Dicarboxy(2,2's-dimethylpropyl)-4,4-diphenylbutadiene,

2,4-Bis[5-1(dimethylpropyl)benzoxazol-2-yl-(4-phenyl)imino]-6-(2-ethylhexyl)imino-1,3,5-triazine,

Octyl 5-N,N-diethylamino-2-phenylsulphonyl-2,4-pentadienoate, and their mixtures.

The additional organic screening agents in accordance with the invention generally represent from 0.1 to 30%, preferably from 1 to 25%, of the total weight of the composition.

Of course, one skilled in the art will take care to select the optional additional screening agent or agents and/or their amounts such that the advantageous properties intrinsically associated with the compositions in accordance with the invention are not, or not substantially, detrimentally affected by the envisaged addition or additions.

The compositions according to the invention can also comprise agents for the artificial tanning and/or browning of the skin (self-tanning agents) and more particularly dihydroxyacetone (DHA). These are preferably present in amounts ranging from 0.1 to 10% by weight, with respect to the total weight of the composition.

The aqueous compositions in accordance with the present invention can additionally comprise conventional cosmetic adjuvants selected in particular from among fatty substances, organic solvents, ionic or nonionic and hydrophilic or lipophilic thickeners, softening agents, humectants, opacifiers, stabilizing agents, emollients, silicones, antifoaming agents, fragrances, preservatives, anionic, cationic, nonionic, zwitterionic or amphoteric surfactants, active principles, fillers, polymers, propellants, basifying or acidifying agents or any other ingredient commonly used in the cosmetics and/or dermatological field.

Of course, one skilled in the art will take care to select the optional additive or additives and/or their amounts such that the advantageous properties intrinsically associated with the compositions in accordance with the invention are not, or not substantially, detrimentally affected by the envisaged addition or additions.

The fatty substances can be composed of an oil or a wax, other than the nonpolar waxes as defined above, or their mixtures. The term “oil” means a compound which is liquid at ambient temperature. The term “wax” means a compound which is solid or substantially solid at ambient temperature and which has a melting point generally of greater than 35° C.

Exemplary oils are mineral oils (liquid paraffin); vegetable oils (sweet almond, macadamia, blackcurrant seed or jojoba oil); synthetic oils, such as perhydrosqualene, fatty alcohols, fatty amides (such as isopropyl lauroyl sarcosinate, marketed under the trademark “Eldew SL-205” by Ajinomoto), fatty acids or esters (such as C₁₂-C₁₅ alkyl benzoate, marketed under the trademark “Finsolv TN” or “Witconol TN” by Witco, 2-ethylphenyl benzoate, such as the commercial product marketed under the trademark X-Tend 226® by ISP, octyl palmitate, isopropyl lanolate, triglycerides, including those of capric/caprylic acids, or dicaprylyl carbonate, marketed under the trademark “Cetiol CC” by Cognis), or oxyethylenated or oxypropylenated fatty esters and ethers; silicone oils (cyclomethicone, polydimethylsiloxanes or PDMSs); fluorinated oils; or polyalkylenes.

Exemplary waxy compounds are carnauba wax, beeswax, hydrogenated castor oil, polyethylene waxes and polymethylene waxes, such as that marketed under the trademark Cirebelle 303 by Sasol.

Exemplary organic solvents are lower alcohols and polyols. The latter can be selected from glycols and glycol ethers, such as ethylene glycol, propylene glycol, butylene glycol, dipropylene glycol or diethylene glycol.

Exemplary hydrophilic thickeners include carboxyvinyl polymers, such as the Carbopols (Carbomers) and the Pemulens (acrylate/C₁₀-C₃₀-alkyl acrylate copolymer); polyacrylamides, such as, for example, the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by Seppic; optionally crosslinked and/or neutralized polymers and copolymers of 2-acrylamido-2-methylpropanesulphonic acid, such as the poly(2-acrylamido-2-methylpropanesulphonic acid) marketed by Hoechst under the trademark “Hostacerin AMPS” (CTFA name: ammonium polyacryloyldimethyl taurate) or Simulgel 800, marketed by Seppic (CTFA name: sodium polyacryloyldimethyl taurate/polysorbate 80/sorbitan oleate); copolymers of 2-acrylamido-2-methylpropanesulphonic acid and of hydroxyethyl acrylate, such as Simulgel NS and Sepinov EMT 10, marketed by Seppic; cellulose derivatives, such as hydroxyethylcellulose; polysaccharides and in particular gums, such as xanthan gum; and their mixtures.

Exemplary lipophilic thickeners include synthetic polymers, such as the poly(C₁₀-C₃₀ alkyl acrylates) marketed under the trademarks “Intelimer IPA 13-1” and “Intelimer IPA 13-6” by Landec, or of modified clays, such as hectorite and its derivatives, such as the products marketed under the Bentone names.

Exemplary active principles include:

vitamins (C, K, PP, and the like) and their derivatives or precursors, alone or as mixtures;

agents for combating pollution and/or agents for combating free radicals;

depigmenting agents and/or propigmenting agents;

antiglycation agents;

soothing agents;

NO-synthase inhibitors;

agents which stimulate the synthesis of dermal or epidermal macromolecules and/or which prevent their decomposition;

agents which stimulate the proliferation of fibroblasts;

agents which stimulate the proliferation of keratinocytes;

muscle-relaxing agents;

tightening agents;

mattifying agents;

keratolytic agents;

desquamating agents;

moisturizing agents;

antiinflammatory agents;

agents which act on the energy metabolism of the cells;

insect repellents;

substance P or substance CRGP antagonists;

agents for combating hair loss and/or for the regrowth of the hair;

antiwrinkle agents.

The compositions according to the invention can be formulated according to techniques well known to one skilled in the art. They can in particular be provided in the form of a simple or complex (O/W, W/O, O/W/O or W/O/W) emulsion, such as a cream or a milk, or of a cream gel; in the form of an aqueous gel; in the form of a lotion. They can optionally be packaged as an aerosol and be provided in the foam or spray form.

Preferably, the compositions according to the invention are provided in the form of an oil-in-water or water-in-oil emulsion.

The emulsions generally comprise at least one emulsifier selected from amphoteric, anionic, cationic or nonionic emulsifiers, employed alone or as a mixture. The emulsifiers are appropriately selected according to the emulsion to be obtained (W/O or O/W emulsion). The emulsions can also comprise other types of stabilizing agents, such as, for example, fillers or gelling or thickening polymers.

Exemplary emulsifying surfactants which can be used for the preparation of the W/O emulsions include sorbitan, glycerol or sugar alkyl esters or ethers; silicone surfactants, such as dimethicone copolyols, for example the mixture of cyclomethicone and of dimethicone copolyol marketed under the trademark “DC 5225 C” by Dow Corning, and alkyl dimethicone copolyols, such as lauryl methicone copolyol, marketed under the trademark “Dow Corning 5200 Formulation Aid” by Dow Corning; cetyl dimethicone copolyol, such as the product marketed under the trademark Abil EM 90R by Goldschmidt, and the mixture of cetyl dimethicone copolyol, of polyglycerol (4 mol) isostearate and of hexyl laurate marketed under the trademark Abil WE 09 by Goldschmidt. It is also possible to add thereto one or more coemulsifiers which, advantageously, can be selected from the group consisting of polyol alkyl esters.

Particularly, exemplary polyol alkyl esters are polyethylene glycol esters, such as PEG-30 dipolyhydroxystearate, such as the product marketed under the trademark Arlacel P135 by ICI.

Exemplary esters of glycerol and/or of sorbitan include polyglycerol isostearate, such as the product marketed under the trademark Isolan GI 34 by Goldschmidt; sorbitan isostearate, such as the product marketed under the trademark Arlacel 987 by ICI; glycerol sorbitan isostearate, such as the product marketed under the trademark Arlacel 986 by ICI, and their mixtures.

Exemplary, for the 0/W emulsions, are, as emulsifiers, nonionic emulsifiers, such as oxyalkylenated (more particularly polyoxyethylenated) esters of fatty acids and of glycerol; oxyalkylenated esters of fatty acids and of sorbitan; oxyalkylenated (oxyethylenated and/or oxypropylenated) fatty acid esters, such as the PEG-100 stearate/glyceryl stearate mixture marketed, for example, by ICI under the trademark Arlacel 165; oxyalkylenated (oxyethylenated and/or oxypropylenated) fatty alcohol ethers; sugar esters, such as sucrose stearate; ethers of fatty alcohol and of sugar, in particular alkyl polyglucosides (APG), such as decyl glucoside and lauryl glucoside, for example marketed by Henkel under the respective names Plantaren 2000 and Plantaren 1200, cetearyl glucoside, optionally as a mixture with cetearyl alcohol, for example marketed under the trademark Montanov 68 by Seppic, under the trademark Tegocare CG90 by Goldschmidt and under the trademark Emulgade KE3302 by Henkel, and arachidyl glucoside, for example in the form of the mixture of arachidyl and behenyl alcohols and of arachidyl glucoside marketed under the trademark Montanov 202 by Seppic. According to a specific embodiment of the invention, the mixture of the alkyl polyglucoside as defined above with the corresponding fatty alcohol can be in the form of a self-emulsifying composition, for example as disclosed in WO-A-92/06778.

More particularly other emulsion stabilizers include polymers of isophthalic acid or sulphoisophthalic acid and in particular the phthalate/sulphoisophthalate/glycol copolymers, for example the diethyleneglycol/phthalate/isophthalate/1,4-cyclohexanedimethanol copolymer (INCI name: Polyester-5) marketed under the trademarks “Eastman AQ polymer” (AQ35S, AQ38S, AQ55S, AQ48 Ultra) by Eastman Chemical.

When an emulsion is provided, the aqueous phase of the latter can comprise a nonionic vesicular dispersion prepared according to known methods (Bangham, Standish and Watkins, J. Mol. Biol., 13, 238 (1965), FR-2,315,991 and FR-2,416,008).

The compositions according to the invention have applications in a large number of treatments, in particular cosmetic treatments, of the skin, lips and hair, including the scalp, in particular for protecting and/or caring for the skin, lips and/or hair and/or for making up the skin and/or lips.

The present invention also features formulation of the subject compositions into products for the cosmetic treatment of the skin, lips, nails, hair, eyelashes, eyebrows and/or scalp, in particular of care products, sun protection products and makeup products.

The cosmetic compositions according to the invention can, for example, be used as makeup products.

The cosmetic compositions according to the invention can, for example, be used as care product and/or sun protection product for the face and/or body with a liquid to semi-liquid consistency, such as milks, relatively smooth creams, cream gels or pastes. They can optionally be packaged in an aerosol and be provided in the foam or spray form.

The compositions according to the invention in the form of vaporizable fluid lotions in accordance with the invention are applied to the skin or hair in the form of fine particles by means of pressurizing devices. The devices in accordance with the invention are well known to a person skilled in the art and comprise non-aerosol pumps or “atomizers”, aerosol containers comprising a propellant and aerosol pumps using compressed air as propellant. The latter are disclosed in U.S. Pat. Nos. 4,077,441 and 4,850,517.

The compositions packaged as an aerosol in accordance with the invention generally comprise conventional propellants, such as, for example, hydrofluorinated compounds, dichlorodifluoromethane, difluoroethane, dimethyl ether, isobutane, n-butane, propane or trichlorofluoromethane. They are preferably present in amounts ranging from 15 to 50% by weight, with respect to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES 1 to 4

### 1. Photostability of the Dibenzoylmethane

The photostabilizing effect of the 2-pyrrolidinone-4-carboxy ester compounds of formula (I) in accordance with the invention was evaluated with respect to the dibenzoylmethane derivative: Butyl Methoxy Dibenzoylmethane (avobenzone), marketed under the trademark “Parsol 1789” by Roche Vitamins (Examples 1, 2, 3 and 4).

The photostabilizing effect of these 2-pyrrolidinone-4-carboxy ester derivatives was also compared with that of Isopropyl Lauroyl Sarcosinate (Example A) of formula:

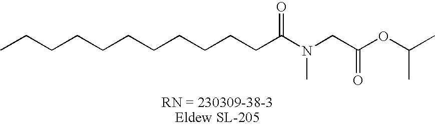

Example B comprises neither pyrrolidone-4-carboxy ester compound nor isopropyl lauroyl sarcosinate.

| Phase | Ingredients | A | B | 1 | 2 | 3 | 4 |
|---|---|---|---|---|---|---|---|
| Fatty | C₁₂-C₁₅ Alkyl | 10.0 | 10.0 | 10.0 | 10.0 | 10.0 | 10.0 |
| | benzoate | | | | | | |
| | Cetearyl | 7.5 | 7.5 | 7.5 | 7.5 | 7.5 | 7.5 |
| | alcohol | | | | | | |
| | and cetearyl | | | | | | |
| | glucoside | | | | | | |
| | Butyl Methoxy | 1.0 | 1.0 | 1.0 | 1.0 | 1.0 | 1.0 |
| | Dibenzoyl- | | | | | | |
| | methane | | | | | | |
| | Isopropyl | 10.0 | — | — | — | — | — |
| | Lauroyl | | | | | | |
| | Sarcosinate | | | | | | |
| | Compound j | — | — | 10.0 | — | — | — |
| | Compound l | — | — | — | 10.0 | — | — |
| | Compound m | — | — | — | — | 10.0 | — |
| | Compound n | 5.0 | 5.0 | 5.0 | 5.0 | 5.0 | 10.0 |
| Aqueous | Water | q.s. | q.s. | q.s. | q.s. | q.s. | q.s. |
| | | for | for | for | for | for | for |
| | | 100 | 100 | 100 | 100 | 100 | 100 |

Principle of the Method:

The percentage of loss of dibenzoylmethane derivative brought about by the exposure to a solar simulator of a formulation spread as films with a thickness of approximately 20 μm is measured. Evaluation is carried out by HPLC analysis of the screening agent in solution, after extraction of the films, by comparing irradiated and nonirradiated samples.

Equipment and Method:

Solar simulator: Oriel 1000W device equipped with a 4-inch outlet, having an 81017 filter and a dichroic mirror. The samples are exposed in a horizontal position. The UV-meter used is the Osram Centra device equipped with 2 reading heads, one for UV-A radiation and the other for UV-B radiation. The simulator/UV-meter assembly is calibrated annually by spectroradiometry. The irradiance measurements are carried out at the beginning and at the end of exposure by placing the reading heads at the position of the sample. The irradiances are:

0.35-0.45 mW/cm² in UV-B radiation

16-18 mW/cm² in UV-A radiation.

The residual Butyl Methoxy Dibenzoylmethane is measured by chromatography: HPLC line with diode array detector. The 2-pyrrolidinone-4-carboxy ester derivatives are introduced into the common vehicle defined above at a concentration of 10% in the presence of 1% of Butyl Methoxy Dibenzoylmethane with the composition: The loss of Butyl Methoxy Dibenzoylmethane (avobenzone) after irradiation in the same vehicle not comprising compound of formula (I) (Composition B) is also measured.

Conducting the Photostability Tests:

Approximately 20 mg of each formulation tested are spread over a surface area of 10 cm² on a ground face of a fused silica disc. The exact amount is determined by weighing. 3 films are exposed to the solar simulator and 3 others act as a control. The samples are exposed 3 by 3 to the light of the solar simulator for a time sufficient to deliver a UV-A dose equal to 12J corrected for the sensitivity of the UV-meter, corresponding to the decomposition of approximately 50% of the Butyl Methoxy Dibenzoylmethane (avobenzone) in the absence of photoprotective agent. At the end of the exposure, each support disc is introduced into a 600 ml container with 10 ml of an appropriate solvent (generally EtOH); the assembly is placed in an ultrasonic bath for 5 minutes. The solution is subsequently transferred into flasks suitable for the vehicle compatible with the HPLC analytical device used. The analytical conditions can be adjusted according to the active principle tested. The residual levels are calculated from the means obtained with regard to the irradiated and nonirradiated samples, as described below:

TABLE I

| | | Residual % level of |
|---|---|---|
| Examples | Amidated compound | avobenzone |
| Example A | Eldew SL-205 | 64.2 ± 0.45 |
| Example B | none | 19.8 ± 0.8 |
| Example 1 | Compound (j) | 72.8 ± 2.0 |
| Example 2 | Compound (l) | 74.5 ± 2.7 |
| Example 3 | Compound (m) | 78.5 ± 1.4 |
| Example 4 | Compound (n) | 70.9 ± 1.3 |

The 2-pyrrolidinone-4-carboxy ester compounds of formulae (j), (l), (m) and (n) of Examples 1 to 4 according to the invention have a photostabilizing effect with regard to the dibenzoylmethane derivative which is better than that obtained with Isopropyl Lauroyl Sarcosinate.

### 2. Formulation Stability of the Compositions

The thermal stabilities of Examples 1, 2, 3 and 4 corresponding to the derivatives of the invention are compared with formulation C, which is identical to formulation A but which comprises, in place of Isopropyl Lauroyl Sarcosinate, the amidated compound ethyl N-butyl-N-acetylaminopropionate (R3535, Merck). The appearance of each formulation after storing in an oven at 45° C. for 2 months is observed with the naked eye (macroscopic appearance).

| | Macroscopic and |
|---|---|
| | microscopic appearance at |
| Compositions | a time of 2 months at 45° C. |
| Formulation 1 comprising compound (j) | The emulsion remains stable |
| | and homogeneous |
| Formulation 2 comprising compound (l) | The emulsion remains stable |
| | and homogeneous |
| Formulation 3 comprising compound (m) | The emulsion remains stable |
| | and homogeneous |
| Formulation 4 comprising compound (n) | The emulsion remains stable |
| | and homogeneous |
| Formulation C comprising ethyl N- | Phase separation is observed |
| butyl-N-acetylaminopropionate (R3535) | |

It is clearly observed that, compared with ethyl N-butyl-N-acetylaminopropionate (R3535) as described in EP 717 982, compounds (j), (l), (m) and (n) of formula (I) according to the invention make it possible to obtain a good stable dispersion of the oil in the composition and, at the end, good formulation stability.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A photostable, topically applicable cosmetic composition comprising at least one UV-screening system, which comprises:
(a) at least one dibenzoylmethane compound, and
(b) at least one 2-pyrrolidinone-4-carboxy ester compound having the following formula (I): 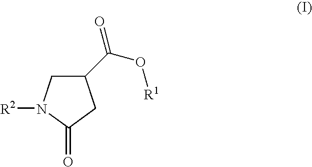
in which:
R¹ is a linear or branched C₁-C₂₀ alkyl radical, and
R² is a linear or branched C₁-C₂₀ alkyl radical optionally comprising a C₅-C₆ ring member, the phenyl radical, the benzyl radical or the phenethyl radical, formulated into a topically applicable, cosmetically acceptable vehicle therefor.

2. The photostable cosmetic composition as defined by claim 1, wherein the at least one compound of formula (I) is selected from among the following compounds (a) to (oo):

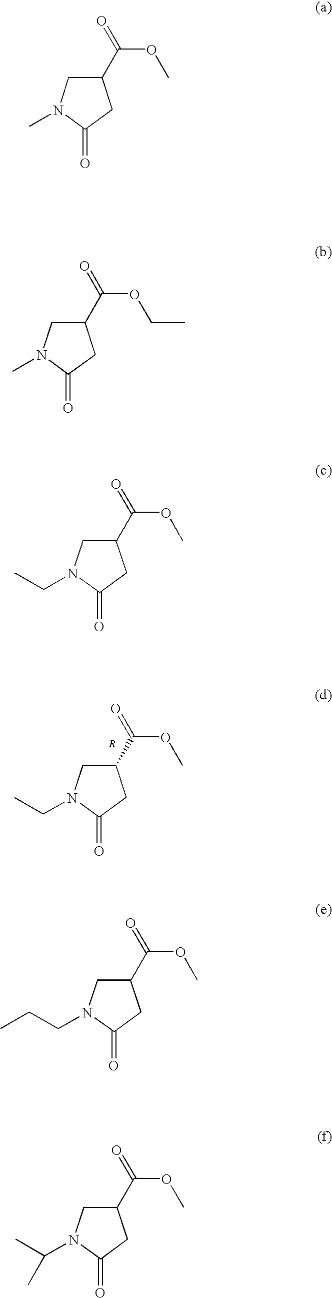

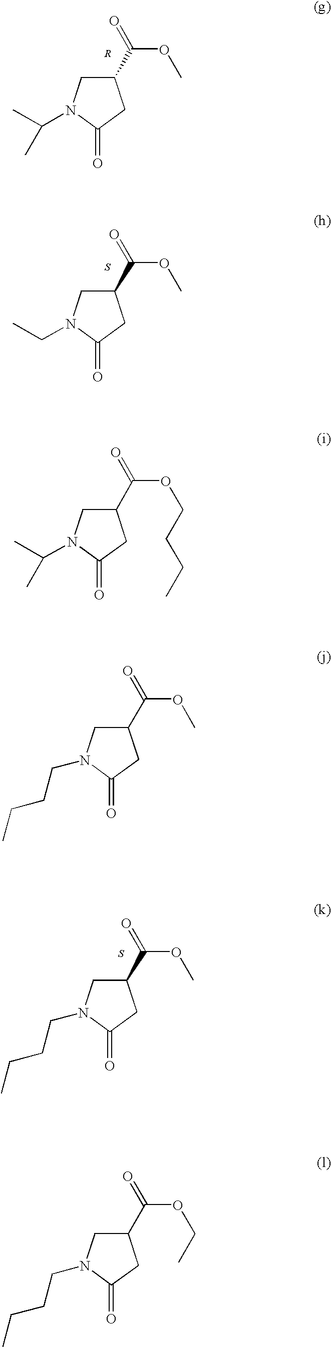

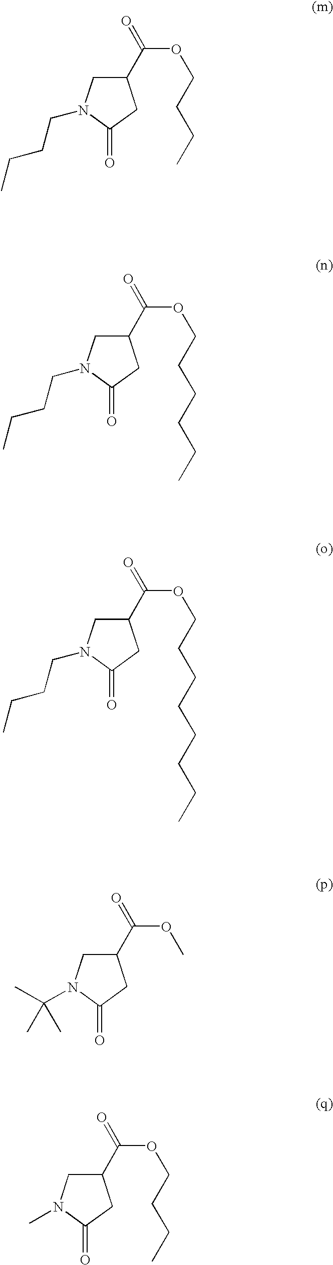

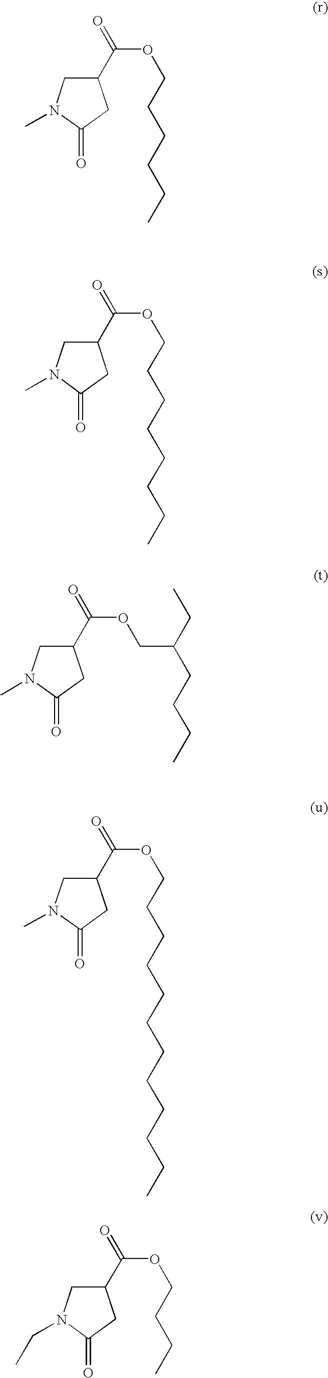

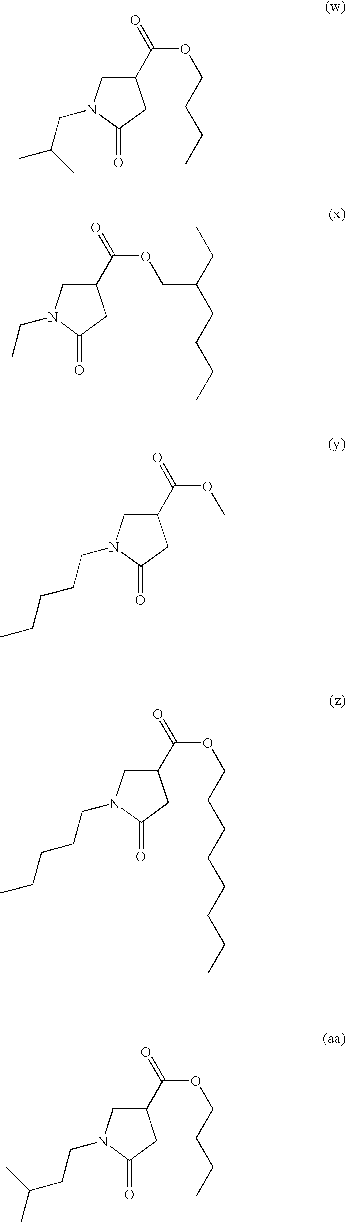

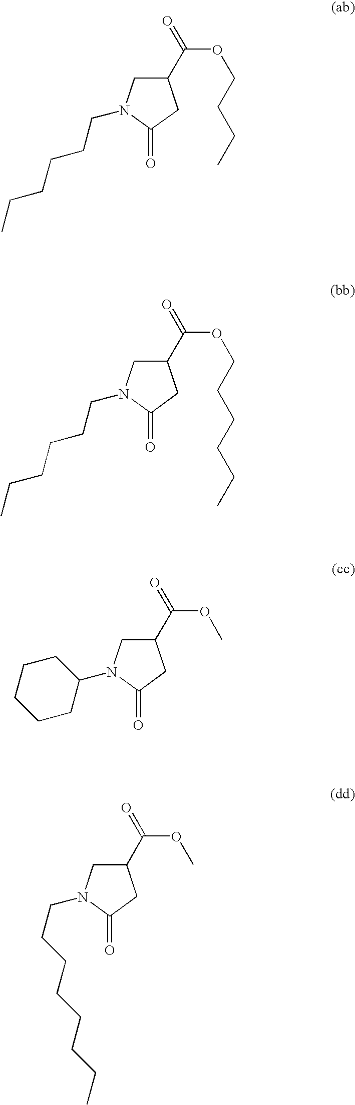

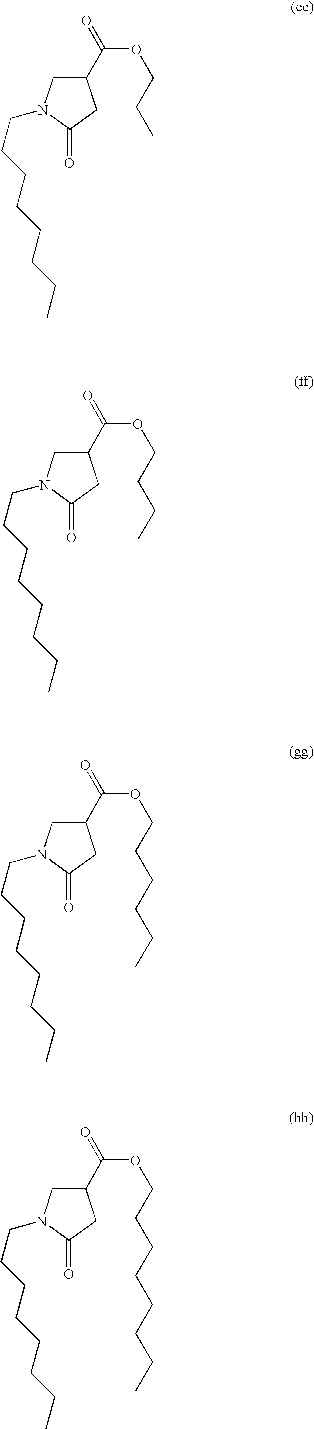

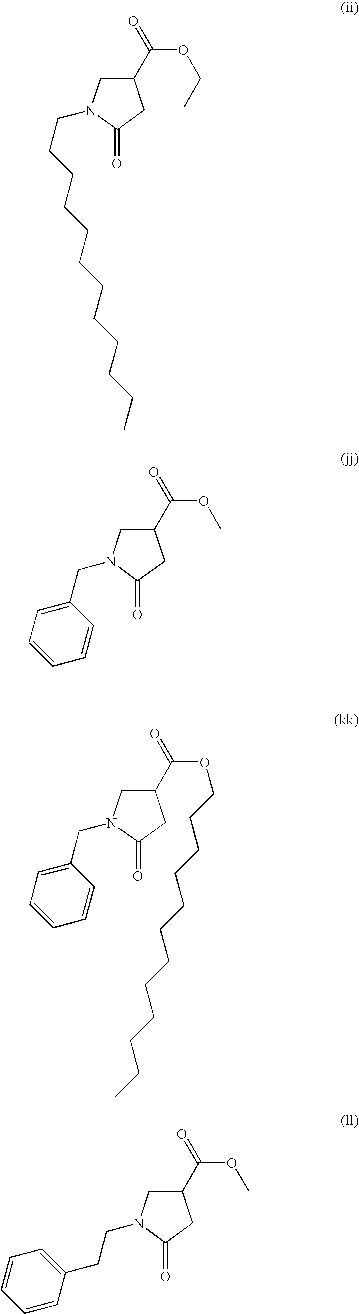

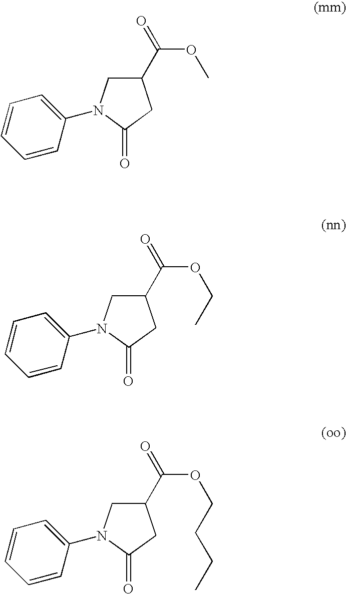

3. The photostable cosmetic composition as defined by claim 2, wherein the at least one compound of formula (I) is selected from among the compounds:

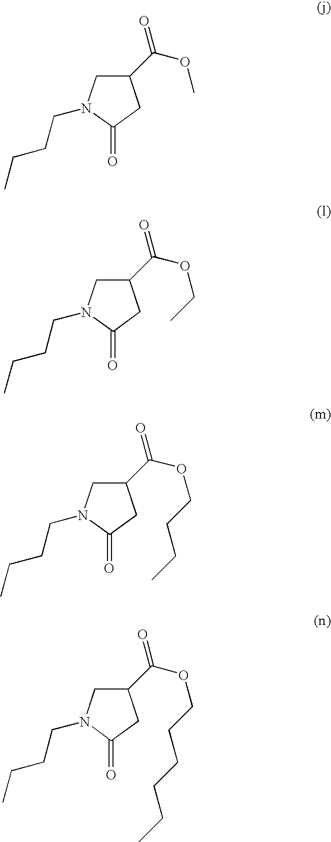

4. The photostable cosmetic composition as defined by claim 1, wherein said at least one dibenzoylmethane compound comprises 4-(tert-butyl)-4's-methoxydibenzoylmethane or Butyl Methoxy Dibenzoylmethane of the following formula:

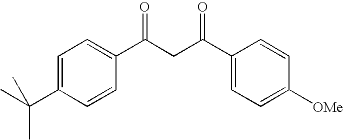

5. The photostable cosmetic composition as defined by claim 1, further comprising at least one silicated s-triazine compound substituted by two aminobenzoate or aminobenzamide groups and having the following general formula (VI) or one of the tautomeric forms thereof: 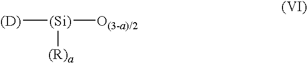
in which:
the radicals R¹, which may be identical or different, are each a linear or branched C₁-C₃₀ alkyl radical which is optionally halogenated or unsaturated,
a C₆-C₁₂ aryl radical, a C₁-C₁₀ alkoxy radical, a hydroxyl radical or the trimethylsilyloxy group;
a=0 to 3; in addition to the structural units of formula -A-(Si)(R)ₐ(O)_{(3-a)/2}, the organosiloxane can comprise units of formula: (R)_{b}—(Si)(O)_{(4-b)/2}, in which:
R has the same definition as in the formula (II) and b=1, 2 or 3;
the group (D) is an s-triazine compound of the following formula (VII): 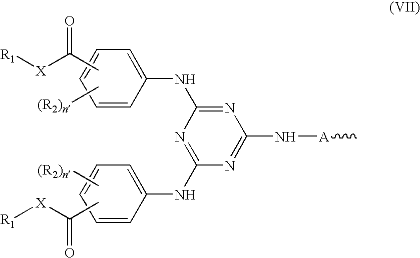
wherein:
X is —O— or —NR₃₋, with R₃ representing hydrogen or a C₁-C₅ alkyl radical,
R₁ is a linear or branched C₁-C₃₀ alkyl radical which is optionally unsaturated and which can comprise a silicon atom, a C₅-C₂₀ cycloalkyl group, optionally substituted by 1 to 3 linear or branched C₁-C₄ alkyl radicals, the —(CH₂CHR₄—O)ₘR₅ group or the
H₂—CH(OH)—CH₂—O—R₆ group,
R₄ is hydrogen or methyl; with the proviso that the (C═O)XR₁ group may be in the ortho, meta or para position with respect to the amino group,
R₅ is hydrogen or a C₁-C₈ alkyl group,
R₆ is hydrogen or a C₄-C₈ alkyl group,
m is an integer ranging from 2 to 20,
n's=0 to 2,
the radicals R₂, which may be identical or different, are each a hydroxyl radical, a linear or branched C₁-C₈ alkyl radical or a C₁-C₈ alkoxy radical, with the proviso that two adjacent R₂ groups on the same aromatic nucleus may together form an alkylidenedioxy group in which the alkylidene radical comprises 1 or 2 carbon atoms,
A is a divalent radical selected from methylene, —[CH(Si(CH₃)₃)]—, ethylene or a radical corresponding to one of the following formulae (VIII), (IX), (X) or (XI): 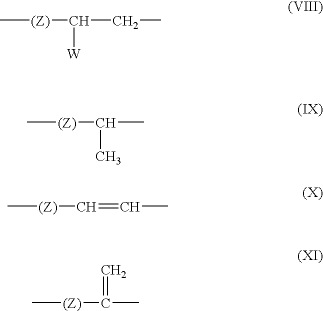
in which:
Z is a saturated or unsaturated and linear or branched C₁-C₁₀ alkylene diradical which is optionally substituted by a hydroxyl radical or oxygen atoms and which can optionally comprise an amino group, and
W is a hydrogen atom, a hydroxyl radical or a saturated or unsaturated and linear or branched C₁-C₈ alkyl radical.

6. The photostable cosmetic composition as defined by claim 5, comprising a compound of formula (VI) which is 2,4-bis(n-butyl 4's-aminobenzoate)-6-[(3-{1,3,3,3-tetramethyl-1-[(trimethylsilyl)oxy]disiloxanyl}propyl)amino]-s-triazine of formula (5):

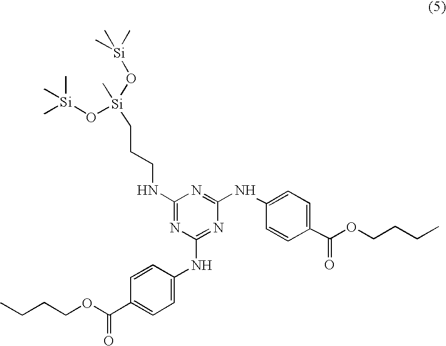

7. The photostable cosmetic composition as defined by claim 1, further comprising other organic or inorganic screening agents active in the UV-A and/or UV-B regions which are water-soluble or fat-soluble or else insoluble in the conventional cosmetic solvents.

8. The photostable cosmetic composition as defined by claim 7, comprising additional organic screening agents selected from among anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from a-alkylstyrene; 4,4-diarylbutadienes; merocyanine derivatives and mixtures thereof.

9. The photostable cosmetic composition as defined by claim 8, comprising organic UV-screening agent or agents selected from among the following compounds:
Ethylhexyl Methoxycinnamate,
Ethylhexyl Salicylate,
Homosalate,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor,
Terephthalylidene Dicamphor Sulfonic Acid,
Disodium Phenyl Dibenzimidazole Tetrasulfonate,
Methylene Bis-Benzotriazolyl Tetramethylbutylphenol,
Bis-Ethylhexyloxyphenol Methoxyphenyl Triazine,
Ethylhexyl Triazone,
Diethylhexyl Butamido Triazone,
2,4,6-Tris(dineopentyl 4's-aminobenzalmalonate)-s-triazine,
2,4,6-Tris(diisobutyl 4's-aminobenzalmalonate)-s-triazine,
2,4-Bis(dineopentyl 4's-aminobenzalmalonate)-6-(n-butyl 4's-aminobenzoate)-s-triazine,
2,4,6-Tris(biphenyl-4-yl)-1,3,5-triazine,
2,4,6-Tris(terphenyl)-1,3,5-triazine,
Drometrizole Trisiloxane,
Polysilicone-15,
1,1-Dicarboxy(2,2's-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine,
Octyl 5-N,N-diethylamino-2-phenylsulphonyl-2,4-pentadienoate,
and mixtures thereof.

10. The photostable cosmetic composition as defined by claim 7, wherein said additional inorganic screening agents comprise coated or uncoated metal oxide pigments.

11. The photostable cosmetic composition as defined by claim 10, comprising coated or uncoated metal oxide pigments having a mean primary particle size of from 5 nm to 100 nm.

12. The photostable cosmetic composition as defined by claim 10, comprising coated or uncoated metal oxide pigments selected from among pigments formed of titanium oxide (amorphous or crystallized in the rutile and/or anatase form), iron oxide, zinc oxide, zirconium oxide or cerium oxide or mixtures thereof.

13. The photostable cosmetic composition as defined by claim 1, formulated as an oil-in-water or water-in-oil emulsion.

14. A method for improving the chemical stability with regard to UV radiation of at least one dibenzoylmethane derivative as defined by claim 1, comprising combining said at least one dibenzoylmethane derivative with an effective amount of at least one 2-pyrrolidinone-4-carboxy ester compound of formula (I) as also defined therein.

15. A regime or regimen for photoprotecting the skin against the damaging effects of UV-radiation, comprising topically applying thereon a thus effective amount of the photostable cosmetic composition as defined by claim 1.
